(19) United States
(12) Patent Application Publication
(10) Pub. No.: US 2022/0145247 A1
(43) Pub. Date: May 12, 2022

(54) GENERATION OF HUMAN PLURIPOTENT STEM CELL DERIVED ARTIFICIAL TISSUE STRUCTURES WITHOUT THREE DIMENSIONAL MATRICES

(71) Applicant: MILTENYI BIOTEC B.V. & CO. KG, Bergisch Gladbach (DE)

(72) Inventors: Kristin BECKER, Bergisch Gladbach (DE); Dominik ECKARDT, Bergisch Gladbach (DE); Andreas BOSIO, Bergisch Gladbach (DE); Sebastian KNÖBEL, Bergisch Gladbach (DE); David Joel AGORKU, Bergisch Gladbach (DE); Olaf Thorsten HARDT, Bergisch Gladbach (DE)

(73) Assignee: MILTENYI BIOTEC B.V. & CO. KG, Bergisch Gladbach (DE)

(21) Appl. No.: 17/430,131

(22) PCT Filed: Feb. 10, 2020
(86) PCT No.: PCT/EP2020/053237

§ 371 Date: Aug. 11, 2021

(30) Foreign Application Priority Data
Feb. 11, 2019 (EP) 19156450.9

(51) Int. Cl. C12N 5/079; C12N 5/071

(52) U.S. Cl. CPC C12N 5/0618; C12N 5/0697; C12N 2500/34; C12N 2501/15; C12N 2533/78; C12N 2501/415; C12N 2506/02; C12N 2513/00; C12N 2501/16

(57) ABSTRACT

The present invention provides a differentiation medium for differentiation and expansion of a multicellular aggregation in suspension derived from human pluripotent stem cells that has been induced to differentiate to an artificial tissue structure such as artificial neural tissue, said medium comprising a basal medium for animal or human cells, wherein said differentiation medium has a viscosity between 1.7 mPa*s and 1500 mPa*s. Said viscosity is achieved by the presence of a viscosity enhancer such as methyl cellulose, carboxymethyl cellulose, or hydroxy ethyl cellulose in said differentiation medium. Also disclosed are an in-vitro method for obtaining artificial neural tissue and a kit comprising said differentiation medium.

### FIELD OF INVENTION

The present invention relates to the field of in vitro generation of artificial tissue structures such as organoids e.g. brain organoids derived from human pluripotent stem cells, in particular to a differentiation medium with defined viscosity allowing for the generation of said artificial tissue structures, e.g. said organoids independent of extracellular matrices.

### BACKGROUND OF THE INVENTION

Depending on the method used for the generation of human pluripotent stem cell (PSC) derived artificial tissue structures, different levels of tissue complexity can be modeled. One can distinguish between two major 3 dimensional (3 D) model systems: spheroids and organoids. While spheroid structures are considered as less complex and a random mixture of cells and cell types, organoids can recapitulate very complex tissue architectures close to the original (in vivo) organ structure and function. Both 3 D structures can be generated from human pluripotent stem cells. One of the most prominent examples of the generation of 3 D structures, are among others (e.g. artificial kidney, heart and retinal tissues) the generation of human brain organoids (artificial neural tissues). Several different protocols have been published to generate different levels of structural complexity in 3D. The most prominent state of the art protocols for the generation of cerebral organoids were published by Lancaster et al. (2013, Nature: 501:373) and Quian et al. (2016, Cell:165:1238; 2018, Nature Protocols, 13:565) and in WO2014090993A1. They describe the stepwise differentiation of human pluripotent stem cells along the developmental pathway towards the formation of brain organoids. All protocols include a Matrigel™ (Corning) based embedding step. It is widely believed that Matrigel™ embedding promotes self-organization of the brain organoid. Moreover, it is supposed to play a role in neuroepithelia expansion and ventricle formation.

Moreover, protocols for cortical spheres have been described e.g. by Paca et al. (Nat Methods. 2015; 12(7):671-8). This protocol described the generation of cortical spheres in suspension, without the use of a Matrigel™ embedding step. As a result, the neuroepithelium is less expanded, the size of the progenitor zones is smaller and less structured. These progenitor zones are referred to as neural rosette like structures rather than ventricle like structures. Moreover, they display a less complex tissue architecture, even though the size reached after 2 months in culture is comparable with brain organoids as described by e.g. Lancaster et al., 2013. In summary, both types of in vitro brain modeling systems differ in size and structural complexity. While organoids generate big ventricle like zones comprising tightly packed neural progenitors, cortical spheres are smaller, show smaller, more neural rosette like structures and represent simplified architecture of the brain.

However high similarities of the model system to human brains, concerning tissue architecture and cellular composition, are desired to study human development and neural diseases in detail. For that reason brain organoids are favored for a lot of applications, even though their generation is time consuming and contains several critical steps e.g. embedding of organoids in Matrigel™. Especially this protocol step is time consuming, requires skilled personal, specific lab equipment and impairs development of scale up (number of paralleled experiments)/large scale processes (high volumes). Moreover, Matrigel™ is a non-defined matrix in which the composition of matrix components differs from lot to lot. This might also influence the differentiation efficiency and lead to batch to batch variations, thereby impairing standardization of manufacturing processes

For these reasons there is a need in the art for an improved or alternative differentiation medium for generation of artificial tissue structures (organoids) such as brain organoids derived from human pluripotent stem cells and/or methods for using said differentiation medium, in particular for the generation of artificial neural tissues (brain organoids).

### SUMMARY OF THE INVENTION

Current methods for generation of cerebral or brain organoids comprise 4 steps as for example disclosed in WO2014090993A1:

1) forming a multicellular aggregation of human pluripotent stem cells in a first medium,

2) culturing said multicellular aggregation in a second medium, i.e. a neural induction medium, thereby inducing the multicellular aggregation to differentiate to neural tissue,

3) culturing said differentiated multicellular aggregation in a three-dimensional matrix such as Matrigel™ in a third medium, i.e. a (cerebral organoid) differentiation medium, thereby expanding said cells in a multicellular aggregation, wherein said cells are allowed to differentiate further, and

4) culturing said expanded multicellular aggregation of cells from step 3) in a suspension culture in a fourth medium.

Surprisingly, the inventors now found that the in vitro procedure of generating artificial tissue structures (or organoids) such as brain organoids derived from a multicellular aggregation in suspension, derived from human pluripotent stem cells that have been induced to differentiate, is feasible without a three-dimensional matrix if the three-dimensional matrix is replaced by a certain viscosity of the corresponding medium (in above mentioned process of WO2014090993A1 the third medium). Said artificial tissue structures such as brain organoids may subsequently be further cultured as displayed in step 4 for of the above described process of WO2014090993A1. The viscosity of said differentiation medium can be achieved by addition of a viscosity enhancer to said differentiation medium. The viscosity enhancer may be any substance that can increase the viscosity of a liquid such as a medium and is biocompatible to cells that are contained in such medium. The viscosity enhancer may be a substance that allows to adjust the viscosity of a liquid such as a (cell) medium to a viscosity between 1.7 mPa*s and 1500 mPa*s, between 2 mPa*s and 1400 mPa*s, between 2 mPa*s and 1000 mPa*s, between 2 mPa*s and 500 mPa*s, between 4 mPa*s and 1000 mPa*s, between 4 mPa*s and 500 mPa*s, between 4 mPa*s and 200 mPa*s, between 4 mPa*s and 100 mPa*s, between 6 mPa*s and 80 mPa*s, between 10 mPa*s and 80 mPa*s or between 10 mPa*s and 50 mPa*s. The viscosity enhancer may be for example selected from the group consisting of non-gelling, biocompatible rheology modifiers such as carrageenans, xanthan gum, and cellulose ether derivates such as methyl cellulose, carboxymethyl cellulose, and hydroxy ethyl cellulose, and mixtures thereof.

The cells are now in suspension in the differentiation medium and there is no need to embed the cells into a three-dimensional matrix for generating artificial tissue structures (or organoids) such as brain organoids.

The omission of a complex three-dimensional matrix such as a gel facilitates the generation of an artificial tissue structure such as brain organoids in a standardized, e.g. automated manner, enabling as well for scale up and large-scale manufacturing processes. Further standardization is achieved by removing lot-lot variations of the Matrigel™ from the system and a more easy handling, since no organoid embedding is needed.

Surprisingly, the herein generated brain organoids obtained by the methods as disclosed herein, that use the herein disclosed differentiation medium are similar to those generated with methods of the prior art that include a step of embedding the cells into the three dimensional matrix. Although being similar and therefore comparable to the brain organoids generated by the methods known in the art they are distinctive from them and have benefits as disclosed herein.

### BRIEF DESCRIPTION OF THE DRAWINGS

FIG. 1: Schematic overview of the differentiation procedure.

A single cell suspension of human pluripotent stem cells was seeded in 96 well ultralow attachment plates. 24 h later, EB like structures formed and the first medium (M1) was replaced by neural induction medium (Medium 2). On day 5 early neural tissue-like structures were transferred to 24 well plates and Medium 3 containing the viscosity enhancer was added. On day 15, the developing neural tissue is then transferred to 10 cm dishes (containing cerebral organoid differentiation medium, i.e. Medium 4), which are placed on a shaker. Depending on the desired developmental stage, the organoids can be cultivated >100 days.

FIG. 2: Generation of human brain organoids

Representative transmitted light microscopy pictures of the generation of human brain organoids are shown. On day 1 cells formed round embryoid body like structures, which show clear surrounding and an integrated structure. The black inner core is surrounded by a clear ring. Five days after seeding, the neural tissue still shows a round structure. The inner of the organoid starts to show some small structures. The organoids seem to be less compact. On day 20 and 30 round structures in the inner of the organoids can be observed. These structures represent neural progenitor zones. As the organoids grow older the structure becomes more dense. No inner structures can be detected by light microscopy at this stage of differentiation.

FIG. 3: Characterization of the brain organoids using flow cytometry

The change in neural marker expression during organoid development was measured over the time of development in order to assess the degree of neural induction. The organoids were analyzed on day 5, 15 and day 30 of differentiation. To that end organoids were harvested using the Multi Tissue Dissociation Kit 3™ in order to obtain single cells. FIG. 3A shows exemplary dot plots (Flow cytometry) of analyzed brain organoid cultures on day 5 and 15. Co-expression of the nuclear neural progenitor markers Pax6 and Sox2 is shown. The amount of double-positive cells reaches ˜90% on day 5, indicating a successful neural induction and the presence of a large neural progenitor population. Contrary to that the amount of positive cells in the progenitor population decreases on day 15. Collected data of different experiments are presented in FIG. 3B. The diagram shows high Pax6 expression on day 5, which is subsequently decreased on day 15 and 30 to ˜35%, indicating a decrease in the neural progenitor population. This is in line with the processes taking place during neural development, because the number of neural progenitor cells decreases over time due to derivation of neurons, thus explaining the decrease in progenitor population.

FIG. 4: Characterization of brain organoids on day 30 and 50

FIG. 4A shows the expression of N-cadherin, which is a typical marker for the apical membrane.

The apical membrane is observed in all progenitor zones (ventricle like structures) lining the ventricle. The expression is independent of the analysis time point FIG. 4B: shows the expression of the nuclear located neural progenitor marker Sox2. The expression of Sox2 is mainly observed in cells in close proximity to the ventricles, which correlates with normal neural development. Moreover, a TuJ1 staining is shown. This cytoskeletal marker is detected in early neurons. The arrangement of progenitor markers at the ventricle and a surrounding TuJ1 staining correlates with standard neural developmental processes. A similar cellular structure is also observed on day 50.

FIG. 4C: Further markers, important during neural development, are TBR2 and Pax6. Pax6 labels the nuclei of neural progenitor cells that are localized near to the ventricle. TBR-2 positive cells represent a different neural progenitor population, which is positioned more basally, making up a subventricular zone. FIG. 4D shows the expression of the nuclear cortical plate marker TBR1 and the deep layer neurons. On day 50 both markers can be detected basally of the ventricular zone. As observed in neural development TBR-1 is found at the very basal site representing the developing cortical plate. In contrast to that CTIP2 is found apically of the cortical plate, representing the formation of deep layer neurons.

FIG. 5 Experimental set up to compare the use of Matrigel™ against medium containing the viscosity enhancer

Single cell suspensions of human pluripotent stem cells were seeded in 96 well ultra-low attachment plates. 24 h later EB like structures formed and the first medium was replaced by neural induction medium (Medium 2). On day 5 early neural tissues were transferred to 24 well. For experimental set up using the viscosity enhancer, medium with methyl cellulose was added. For the experimental set up using Matrigel™, organoids were embedded in a Matrigel™ droplet using standard procedures and cultivated in a medium without the viscosity enhancer. On day 15 the developing neural tissue is then transferred to 10 cm dishes (containing cerebral organoid differentiation medium), which are placed onto a shaker. Depending on the desired developmental stage the organoids can be cultivated >100 days.

FIG. 6: Comparison of the use of Matrigel™ against medium containing viscosity enhancer for the generation of brain organoids

Transmitted light microscopy images of organoids generated using the standard method described by Lancaster et al (2013, Nature: 501:373) and Quian et al (2016, Cell:165: 1238; 2018, Nature Protocols, 13:565) and in WO2014090993A1 are shown. Organoids are embedded in Matrigel™ for differentiation and cultivation. A dense organoid structure can be observed. Moreover, some neural outgrowth indicated by arrows can be shown. Some cells seem to migrate into the Matrigel™. A smooth surface cannot be observed. Depending on the batch of organoids, less dense structures and fluid-filled cavities can be observed, indicating partial non-specific differentiation, which cannot be observed when using the medium supplemented with viscosity enhancer.

In contrast to that organoids generated without Matrigel™ but using the viscosity enhancer show a smooth surface without any neural outgrowth or fluid filled cavities.

FIG. 7: Titration of different media viscosities

In order to determine the range of viscosity that supports brain organoid formation, different methyl cellulose viscosities were tested. Transmitted light data (day 30 organoids) obtained from brain organoids cultivated in 0%, 0.25%, 0.5%, 1% or 2% methyl cellulose are shown. The cultivation of organoids without any viscosity enhancer leads to a “lose” structure of the organoids. They become less compact and more fringy. Over time, the majority of these organoids dissolve completely, thus leading to highly decreased yields in organoids. Brain organoids generated by the use of 0.25%-1% Methyl cellulose are more dense and show very compact structures. Moreover, they have a smooth border and some cellular structures within the organoids can be observed. This indicates the successful generation of brain organoids containing typical progenitor zones.

The addition of 2% methyl cellulose to the medium, leads to a highly increased viscosity. The organoids are smaller compared to other conditions. They are very compact and without any visible specific structures inside.

FIG. 8: Tissue clearing of organoids obtained from different methyl cellulose concentrations In order to find out whether progenitor cells formed in the inner core of the organoid, the organoids were stained for the proliferation marker Ki67 and cleared using a tissue clearing procedure based on ethyl cinnamate as organic solvent. The cleared brain organoids were analyzed using confocal microcopy and Z stacks, which were reconstructed to illustrate complete organoids including the ventricle-like zones. In the 0.5% and 1% sample circular ventricle like structures were observed. These structures were found all over the organoid. In contrast to that organoids generated using 2% methyl cellulose do not show a Ki67 positive cells, indicating the absence of ventricle like zones.

FIG. 9: Comparison of different viscosity enhancer based on organoid morphology

Representative transmitted light microscopy pictures of the generation of human brain organoids at day 7 and day 25 are shown. 0.5% methyl cellulose, 0.21% carboxymethyl cellulose and 0.25% hydroxy ethyl cellulose were used as viscosity enhancer in Medium 3. On day 7 organoids in all three conditions show some small structures in the inner parts and bulges at the surface, indicating ongoing differentiation and proliferation. On day 30 round structures in the inner of the organoids can be observed, representing neural progenitor zones. On both days all three conditions look comparable.

FIG. 10: Comparison of different viscosity enhancer based on flow cytometry

Neural marker expression was measured on day 30 of organoid development to compare differentiation efficiency using different viscosity enhancer in Medium 3. To that end organoids were harvested using the Multi Tissue Dissociation Kit 3′ in order to obtain single cells. The expression of the nuclear neural progenitor markers Pax6 and Sox2 and the cytoskeletal marker in early neurons TuJ1 is shown. All three conditions show similar marker expression with an expression between 33-40% Sox2, 10-15% Pax6 and 45-50% TuJ1

### DETAILED DESCRIPTION OF THE INVENTION

In a first aspect the present invention provides a differentiation medium for differentiation and expansion of a multicellular aggregation in suspension derived from human pluripotent stem cells that has been induced to differentiate to an artificial tissue structure, e.g. artificial neural tissue (brain organoid), said medium comprising a basal medium for animal or human cells, wherein said differentiation medium has a viscosity between 1.7 mPa*s and 1500 mPa*s, between 2 mPa*s and 1400 mPa*s, between 2 mPa*s and 1000 mPa*s, between 2 mPa*s and 500 mPa*s, between 4 mPa*s and 1000 mPa*s, between 4 mPa*s and 500 mPa*s, between 4 mPa*s and 200 mPa*s, between 4 mPa*s and 100 mPa*s, between 6 mPa*s and 80 mPa*s, between 10 mPa*s and 80 mPa*s or between 10 mPa*s and 50 mPa*s.

Preferentially, said viscosity is between 4 mPa*s and 100 mPa*s, more preferentially the viscosity is between 6 mPa*s and 80 mPa*s, most preferentially the viscosity is between 10 mPa*s and 80 mPa*s.

Said differentiation medium, wherein said viscosity between 1.7 mPa*s and 1500 mPa*s, between 2 mPa*s and 1400 mPa*s, between 2 mPa*s and 1000 mPa*s, between 2 mPa*s and 500 mPa*s, between 4 mPa*s and 1000 mPa*s, between 4 mPa*s and 500 mPa*s, between 4 mPa*s and 200 mPa*s, between 4 mPa*s and 100 mPa*s, between 6 mPa*s and 80 mPa*s, between 10 mPa*s and 80 mPa*s or between 10 mPa*s and 50 mPa*s is achieved by the presence of a viscosity enhancer in said differentiation medium, therefore said differentiation medium may comprise

i) a basal medium for animal or human cells, and

ii) a viscosity enhancer,

wherein said differentiation medium has a viscosity between 1.7 mPa*s and 1500 mPa*s, between 2 mPa*s and 1400 mPa*s, between 2 mPa*s and 1000 mPa*s, between 2 mPa*s and 500 mPa*s, between 4 mPa*s and 1000 mPa*s, between 4 mPa*s and 500 mPa*s, between 4 mPa*s and 200 mPa*s, between 4 mPa*s and 100 mPa*s, between 6 mPa*s and 80 mPa*s, between 10 mPa*s and 80 mPa*s or between 10 mPa*s and 50 mPa*s.

Said differentiation medium may be without a three-dimensional matrix.

Said viscosity enhancer does not build a three-dimensional matrix in the cell culture medium.

Said viscosity enhancer may be biocompatible for the cells of said differentiation medium.

Said viscosity enhancer may be for example a non-gelling, biocompatible rheology modifier.

Rheology modifiers may be carrageenans, xanthan gum, and cellulose ether derivates such as methyl cellulose, carboxymethyl cellulose, and hydroxy ethyl cellulose, and mixtures thereof.

Said viscosity enhancer may be selected for example from the group of biocompatible rheology modifiers consisting of carrageenans, xanthan gum, and cellulose ether derivates such as methyl cellulose, carboxymethyl cellulose, and hydroxy ethyl cellulose, and mixtures thereof.

Preferentially said viscosity enhancer is methyl cellulose, carboxymethyl cellulose, hydroxy ethyl cellulose, or a combination thereof.

Said viscosity enhancer, wherein said viscosity enhancer is methyl cellulose, carboxymethyl cellulose or hydroxy ethyl cellulose, and wherein the concentration of methyl cellulose, carboxymethyl cellulose, or hydroxy ethyl cellulose is between 0.1% and 2% methyl cellulose, carboxymethyl cellulose, or hydroxy ethyl cellulose in said medium.

Said viscosity enhancer, preferentially methyl cellulose, carboxymethyl cellulose, or hydroxy ethyl cellulose, wherein said viscosity enhancer increases the viscosity of said differentiation medium to a value between 1.7 mPa*s and 1500 mPa*s.

Said differentiation medium may comprise additionally one or more differentiation factors for differentiation of said multicellular aggregation to an artificial tissue structure.

Said one or more differentiation factors may differentiate said multicellular aggregation to artificial neural tissue, to artificial cardiac tissue, to artificial kidney tissue, or artificial retinal tis sue.

Said differentiation medium, wherein said artificial tissue structure may be artificial neural tissue, and wherein said differentiation medium optionally may comprise one or more differentiation factors selected from the group consisting of activator of Wnt signaling and an inhibitor for TGF-beta, activin and nodal signaling pathway.

Surprisingly, said differentiation and expansion of said multicellular aggregation in suspension derived from human pluripotent stem cells that has been induced to differentiate to an artificial neural tissue works in said differentiation medium also without the addition of said one or more differentiation factors.

Said differentiation medium, wherein said artificial tissue structure may be cardiac organoids, and wherein said differentiation medium may comprise one or more differentiation factors selected from the group consisting of Wnt activators and inhibitors, activators of the BMP, activin and bFGF pathway.

Said differentiation medium, wherein said artificial tissue structure may be kidney organoids, and wherein said differentiation medium may comprise one or more differentiation factors selected from the group consisting of Wnt activators and activators of FGF signaling.

Said differentiation medium, wherein said artificial tissue structure may be retinal organoids, and wherein said differentiation medium may comprise one or more differentiation factors selected from the group consisting of Wnt inhibitors and activators of sonic hedgehog pathway.

Said differentiation medium may be used within the method for obtaining artificial neural tissues (brain organoids) as disclosed herein or as disclosed in WO2014090993A1. Of course, in the method as disclosed in WO2014090993A1 said differentiation medium replaces the medium of step 3 (culturing said differentiated multicellular aggregation in a three-dimensional matrix such as Matrigel™)

The steps for methods of obtaining artificial tissue structures (organoids) other than artificial neural tissue (brain organoids), e.g. artificial cardiac tissue (cardiac organoids), artificial kidney tissue (kidney organoids) or artificial retinal tissue (retinal organoids) may vary from the steps of obtaining artificial neural tissues (brain organoids). But all these methods for obtaining artificial tissue structures (organoids) have in common, that the step, wherein a three-dimensional matrix is used can be replaced by the use of the differentiation medium as disclosed herein.

Said pluripotent stem cells may be human embryonic stem cells or human induced pluripotent stem cells.

In a further aspect, the present invention provides an in vitro method for obtaining a brain organoid (an artificial neural tissue) comprising

a) providing a multicellular aggregation of human pluripotent stem cells,

b) culturing said multicellular aggregation in an induction medium (neural induction medium) thereby inducing the multicellular aggregation to differentiate to a brain organoid (an artificial neural tissue),

c) culturing said differentiated multicellular aggregation in suspension in a differentiation medium, wherein said differentiation medium has a viscosity between 1.7 mPa*s and 1500 mPa*s, between 2 mPa*s and 1400 mPa*s, between 2 mPa*s and 1000 mPa*s, between 2 mPa*s and 500 mPa*s, between 4 mPa*s and 1000 mPa*s, between 4 mPa*s and 500 mPa*s, between 4 mPa*s and 200 mPa*s, between 4 mPa*s and 100 mPa*s, between 6 mPa*s and 80 mPa*s, between 10 mPa*s and 80 mPa*s or between 10 mPa*s and 50 mPa*s, thereby expanding the cells in a multicellular aggregation, wherein said cells are allowed to differentiate further.

Said method for obtaining a brain organoid, wherein said method comprises the additional step:

d) culturing said expanded multicellular aggregation of cells from Step c) in a suspension culture.

Said multicellular aggregation of human pluripotent stem cells that may be provided in step a) of said method may be generated in a “medium for generation of multicellular aggregation from human pluripotent stem cells”.

Media for generation of multicellular aggregation from human pluripotent stem cells are well-known in the art and disclosed for example in Eiraku et al (Cell Stem Cell, 2008, 3:519-532), US20110091869, WO2011055855A1 and WO2014090993A1.

Said medium for generation of multicellular aggregation from human pluripotent stem cells (or medium A) may comprise a) a basal medium for animal or human cells, and ii) a Rock inhibitor. The addition of a Rock inhibitor e.g. Thiazovivin, Y27632 is preferred. Such medium is used e.g. in the examples.

Media for induction of multicellular aggregation from human pluripotent stem cells to differentiate to artificial neural tissue (neural induction medium) are well-known in the art and are disclosed for example in Eiraku et al (Cell Stem Cell, 2008, 3:519-532), US20110091869, WO2011055855A1 and WO2014090993A1.

Said neural induction medium (or medium B) of step b) of said method for the differentiation of the multicellular aggregates into artificial neural tissue may comprise i) a basal medium for animal or human cells, ii) an inhibitor for TGF-beta, Activin and Nodal signaling pathway, and iii) a Bone Morphogenetic Protein (BMP) inhibitor.

Said method for obtaining a brain organoid (an artificial neural tissue), wherein said differentiation medium comprises

i) said basal medium for animal or human cells, and

ii) said viscosity enhancer; and optionally

iii) an activator of Wnt signaling and/or an inhibitor for TGF-beta, activin and nodal signaling pathway.

Said culturing human pluripotent stem cells such as iPS cells as multicellular aggregates in said medium for generation of multicellular aggregation from human pluripotent stem cells may be performed for 1-5 days, preferentially for 24 h. In particular this culture step is performed from day 0-1. Induction of artificial neural tissues from these multicellular aggregates in said neural induction medium may be performed for 4-7 days, preferentially for 4 days, e.g. from day 1-4 of differentiation. The culture step of cultivating cells in differentiation medium may be performed for 8-12 days, preferentially 10 days. In particular, said step may be performed from day 5-15. The suspension culture (after culturing cells in differentiation medium containing a viscosity enhancer) in said medium for culturing the expanded multicellular aggregation may be a stirring and/or shaking culture (shaker, bioreactor etc.). Dependent on the development stage said suspension culture may be maintained in said medium for culturing the expanded multicellular aggregation under stirring and/or shaking conditions for up to 100 days or even more days.

The artificial neural tissue (brain organoid) developed by the method as disclosed herein may be e.g. a cerebral organoid, a midbrain organoid or a hindbrain organoid. The development of the kind or type of brain organoid may dependent on the addition or exclusion of different small molecules such as sonic hedgehog leads to the generation of a ventral type of forebrain organoid, while the addition of CHIR and sonic hedgehog leads to caudalization of the brain regions generated in the organoid. Dependent on the combination of small molecules organoids for different brain regions can be generated.

Said method, wherein said differentiation medium comprises a viscosity enhancer that is biocompatible for the cells of said medium as disclosed above, thereby adjusting said viscosity of said differentiation medium between 1.7 mPa*s and 1500 mPa*s, between 2 mPa*s and 1400 mPa*s, between 2 mPa*s and 1000 mPa*s, between 2 mPa*s and 500 mPa*s, between 4 mPa*s and 1000 mPa*s, between 4 mPa*s and 500 mPa*s, between 4 mPa*s and 200 mPa*s, between 4 mPa*s and 100 mPa*s, between 6 mPa*s and 80 mPa*s, between 10 mPa*s and 80 mPa*s or between 10 mPa*s and 50 mPa*s.

Said differentiation medium may be without a three-dimensional matrix.

Said viscosity enhancer does not build a three-dimensional matrix in the cell culture medium.

Preferentially said viscosity enhancer is methyl cellulose, carboxymethyl cellulose, hydroxy ethyl cellulose, or a combination thereof.

Said viscosity enhancer, wherein said viscosity enhancer is methyl cellulose, carboxymethyl cellulose or hydroxy ethyl cellulose, and wherein the concentration of methyl cellulose, carboxymethyl cellulose, or hydroxy ethyl cellulose is between 0.1% and 2% methyl cellulose, carboxymethyl cellulose, or hydroxy ethyl cellulose in said medium.

Said viscosity enhancer, preferentially methyl cellulose, carboxymethyl cellulose, or hydroxy ethyl cellulose, wherein said viscosity enhancer increases the viscosity of said differentiation medium to a value between 1.7 mPa*s and 1500 mPa*s.

Said differentiation medium (Medium C) of step c) of said method may be used for further cell specification and neural epithelia expansion. Said differentiation medium may comprise a basal medium for animal or human cells, wherein said differentiation medium has a viscosity between 1.7 mPa*s and 1500 mPa*s, between 2 mPa*s and 1400 mPa*s, between 2 mPa*s and 1000 mPa*s, between 2 mPa*s and 500 mPa*s, between 4 mPa*s and 1000 mPa*s, between 4 mPa*s and 500 mPa*s, between 4 mPa*s and 200 mPa*s, between 4 mPa*s and 100 mPa*s, between 6 mPa*s and 80 mPa*s, between 10 mPa*s and 80 mPa*s or between 10 mPa*s and 50 mPa*s.

Said differentiation medium, wherein said viscosity between 1.7 mPa*s and 1500 mPa*s, between 2 mPa*s and 1400 mPa*s, between 2 mPa*s and 1000 mPa*s, between 2 mPa*s and 500 mPa*s, between 4 mPa*s and 1000 mPa*s, between 4 mPa*s and 500 mPa*s, between 4 mPa*s and 200 mPa*s, between 4 mPa*s and 100 mPa*s, between 6 mPa*s and 80 mPa*s, between 10 mPa*s and 80 mPa*s or between 10 mPa*s and 50 mPa*s is achieved by the presence of a viscosity enhancer in said differentiation medium, therefore said differentiation medium may comprise

i) a basal medium for animal or human cells, and

ii) a viscosity enhancer,

wherein said differentiation medium has a viscosity between 1.7 mPa*s and 1500 mPa*s, between 2 mPa*s and 1400 mPa*s, between 2 mPa*s and 1000 mPa*s, between 2 mPa*s and 500 mPa*s, between 4 mPa*s and 1000 mPa*s, between 4 mPa*s and 500 mPa*s, between 4 mPa*s and 200 mPa*s, between 4 mPa*s and 100 mPa*s, between 6 mPa*s and 80 mPa*s, between 10 mPa*s and 80 mPa*s or between 10 mPa*s and 50 mPa*s.

Said viscosity enhancer may be biocompatible for the cells of said differentiation medium.

Said viscosity enhancer may be for example a non-gelling, biocompatible rheology modifier.

Rheology modifiers may be carrageenans, xanthan gum, and cellulose ether derivates such as methyl cellulose, carboxymethyl cellulose, and hydroxy ethyl cellulose, and mixtures thereof.

Said viscosity enhancer may be selected for example from the group of biocompatible rheology modifiers consisting of carrageenans, xanthan gum, and cellulose ether derivates such as methyl cellulose, carboxymethyl cellulose, and hydroxy ethyl cellulose, and mixtures thereof.

Preferentially said viscosity enhancer is methyl cellulose, carboxymethyl cellulose, hydroxy ethyl cellulose, or a combination thereof.

Said viscosity enhancer, wherein said viscosity enhancer is methyl cellulose, carboxymethyl cellulose or hydroxy ethyl cellulose, and wherein the concentration of methyl cellulose, carboxymethyl cellulose, or hydroxy ethyl cellulose is between 0.1% and 2% methyl cellulose, carboxymethyl cellulose, or hydroxy ethyl cellulose in said medium.

Said viscosity enhancer, preferentially methyl cellulose, carboxymethyl cellulose, or hydroxy ethyl cellulose, wherein said viscosity enhancer increases the viscosity of said differentiation medium to a value between 1.7 mPa*s and 1500 mPa*s.

Said differentiation medium may comprise additionally one or more differentiation factors for differentiation of said multicellular aggregation to artificial neural tissue.

Said differentiation medium optionally may comprise one or more differentiation factors selected from the group consisting of activator of Wnt signaling and an inhibitor for TGF-beta, activin and nodal signaling pathway.

As mentioned above, said differentiation and expanding of said multicellular aggregation in suspension derived from human pluripotent stem cells that has been induced to differentiate to an artificial neural tissue works in said differentiation medium also without the addition of said one or more differentiation factors.

Therefore, said differentiation medium may be composed of a medium containing a viscosity enhancer as disclosed herein such as methyl cellulose generating a viscosity as disclosed herein. The medium may be further composed of: N2 (transferrin, insulin, Progesterone, Putrescine, Selenite), L-glutamine. For further cell specification and neuroepithelia expansion a Wnt activator e.g. CHIR99021 and/or activator of TGF-β, Activin and Nodal signaling pathway e.g. SB431542 may be added. Such medium is used e.g. in the examples.

Said differentiation medium (or medium C) may be used for further cell specification and neural epithelia expansion (step c of the said method) without the use or the need of a three-dimensional matrix such as Matrigel™ as disclosed e.g. in WO2014090993A1.

Said culturing of said expanded multicellular aggregation of cells from step c) in suspension culture (step d of said method) may be performed in “medium for culturing the expanded multicellular aggregation”. Such media for culturing the expanded multicellular aggregation are well-known in the art and disclosed e.g. in WO2014/090993A1. Said medium for culturing the expanded multicellular aggregation (or medium D) may comprise i) a basal medium for animal or human cells, and ii) retinoic acid and retinol.

Therefore, said medium for culturing the expanded multicellular aggregation may be used for culturing the brain organoids in suspension culture. The medium may be composed e.g. NB21 supplement (MACS® NeuroBrew®-21, Miltenyi Biotec)) or any components thereof. Such medium is used e.g. in the examples.

The suspension culture of step d of the method as disclosed herein (i.e. after culturing cells in differentiation medium containing a viscosity enhancer in said medium for culturing the expanded multicellular aggregation) may be a stirring and/or shaking culture (e.g. a shaker or bioreactor).

Any of the above described media further may contain nutrients, buffers and oxygen. The medium may further comprise growth factors or lack growth factors. Preferred nutrients include a carbohydrate, especially a mono-hexose or mono-pentose, such as glucose or fructose. In a preferred embodiment any media is serum and Xeno free.

Said method for obtaining a brain organoid, wherein said pluripotent stem cells are human induced pluripotent stem cells.

In a further aspect the present method provides the use of a viscosity enhancer for adjusting the viscosity of a cell medium used for obtaining an artificial tissue structure derived from human pluripotent stem cells, wherein said viscosity is between 1.7 mPa*s and 1500 mPa*s, between 2 mPa*s and 1400 mPa*s, between 2 mPa*s and 1000 mPa*s, between 2 mPa*s and 500 mPa*s, between 4 mPa*s and 1000 mPa*s, between 4 mPa*s and 500 mPa*s, between 4 mPa*s and 200 mPa*s, between 4 mPa*s and 100 mPa*s, between 6 mPa*s and 80 mPa*s, between 10 mPa*s and 80 mPa*s or between 10 mPa*s and 50 mPa*s.

In an aspect the present invention provides a brain organoid obtainable by the in-vitro methods for obtaining a brain organoid as disclosed herein.

The brain organoid obtained by said method for obtaining a brain organoid is an artificial neural tissue because said method is performed in vitro and the neural tissue does not reach the complexity of a naturally grown neural tissue.

The brain organoid (artificial neural tissue) obtainable by the methods as disclosed herein resembles the brain organoid known in the art and disclosed for example in WO2014/090993 that needs three-dimensional matrix such as Matrigel™ and that has been characterized as follows:

The brain organoid is comprised of a heterogenous population of cells of at least two different progenitor and neuronal differentiation layers, wherein at least one progenitor layer comprises outer radial glia cells. The brain organoids display a well-organized cerebral cortex. Furthermore, these tissues display several characteristics specific to humans, namely the presence of a substantial outer radial glial population and the organization of extra cortical subventricular zone layers not present in mouse. The presence of outer radial glia cells appears to be one of the most distinguishing features, but of course others exist as well. Eiraku et al. (2008) for example describes that in their culture radial glia of cortical tissues decreased after day 12 and apparently failed to develop into outer radial glia cells, outer radial glia being characterized by their position as well as morphology (lack of an apical connection to the fluid-filled ventricular-like cavity). According to the three-dimensional neural tissue culture of WO2014/090993, the outer radial glia cells are preferably in a progenitor layer, in particular, in a subventricular zone localized basally of the ventricular zone where radial glia reside. The brain organoid disclosed in WO2014/090993 may develop into a differentiated tissue comprising layers of different differentiation grade. In a 3D structure this may be observable as separate sections of the organoid. In preferred embodiments, the culture artificial neural tissue comprises sections from at least two layers. Such a layer may be shaped around a globular tissue body, e.g. a body from which the distinct layer (s) have developed. In particular, the tissue may show a distinctive development of apical and dorsal tissue sections.

The brain organoid disclosed in WO2014/090993 is or resembles cerebral tissue comprising substantially all cells found in the brain or progenitors thereof. Such cells can be identified by selective gene expression markers, which are on a level above the average of not differentiated cells, in particular including confidence intervals. Such markers can be identified by specific antibodies that are used for flow cytometry and immunofluorescence.

Preferably cells of the brain organoids express one or more gene expression markers selected from forebrain markers FoxG1 and Pax6.

The brain organoids can alternatively or in addition be characterized by comprising cells expressing one or more expression markers selected from N-Catherin, Sox2, TuJ1, Pax6 Otx2, FoxG1, Tbr1, Tbr2, Satb2, Ctip2, or any combination thereof.

Preferably the brain organoid comprises cells, which express FoxG1. FoxG1 is expressed in cells of dorsal cortex identity.

Preferably the brain organoid comprises cells, which express Pax6. Pax is expressed in cells of frontal cortex identity.

Preferentially brain organoid comprises cells, which express Sox2 and Pax6 localized near to a ventricle. These markers are expressed in forebrain progenitor populations.

Preferably the brain organoid comprises cells, which express TBR-2. TBR-2 is expressed in intermediate progenitors.

Preferably the brain organoid comprises cells, which express Tuj1. Tuj1 is expressed in cells of a cortical inner fiber layer identity.

Preferably the brain organoid comprises cells, which express Brn2. Brn2 is expressed in cells of a later born neuron (neuron of outer region).

Preferably the brain organoid comprises cells, which express Satb2. Satb2 is expressed in cells of a later born neuron (neuron of outer region).

Preferably the brain organoid comprises cells, which express Ctip2. Ctip2 is expressed in cells of earlier born neuron (neuron of inner region).

Preferably the brain organoid comprises cells, which express TBR-1. TBR-1 is expressed in cells of cortical interneurons within the dorsal cortical plate.

Although the brain organoid obtainable by the method for obtaining brain organoids as disclosed herein has most or many of the features of the cerebral organoid as disclosed in WO2014/090993 in common, some differences exists between these two kinds of organoids. The differences may be traced back to the different methods used for obtaining the organoids. The brain organoids obtained by the method as disclosed herein have less neural outgrowths compared to the brain organoids obtained by the methods known in the art that use ECM (see FIG. 6). The brain organoid obtained by the method as disclosed herein may have at least 20%, 30%, 40%, 50%, 60%, 70%, 80%, 90%. 95%, 99% less neural outgrowths than the brain organoids obtained by the methods known in the art that use three dimensional matrix such as ECM. Preferentially, the brain organoids as disclosed herein may have no neural outgrowths and therefore may have a smooth surface. The brain organoids of the prior art do not have a smooth surface as cells of the organoid invade into the ECM and therefore generate neural outgrowths.

Brain organoids obtained by the method as disclosed herein have benefits compared to the brain organoids obtained by methods of the prior art:
less unspecific neural differentiation and less directed neural migration to the outside of the organoid (FIG. 6)
Organoids remodel neural development more closely since there is no Matrigel™ present during (embryonic) development
Generated neurons stay within the organoid, this might improve neural differentiation and cortical plate development neural layering

It is self-explaining that the brain organoid developed by the methods of the present invention has also a biochemical distinction to the brain organoids developed by the method of the prior art that need the presence of an ECM such as disclosed in WO2014/090993A1. This is e.g. indicative by the missing of a contact area between the cells of the developing organoid as disclosed herein and an ECM (FIG. 6).

In a further aspect the present invention provides a kit comprising a differentiation medium for differentiation and expansion of a multicellular aggregation in suspension derived from human pluripotent stem cells that has been induced to differentiate to an artificial tissue structure, said medium comprising a basal medium for animal or human cells, wherein said differentiation medium has a viscosity between 1.7 mPa*s and 1500 mPa*s, between 2 mPa*s and 1400 mPa*s, between 2 mPa*s and 1000 mPa*s, between 2 mPa*s and 500 mPa*s, between 4 mPa*s and 1000 mPa*s, between 4 mPa*s and 500 mPa*s, between 4 mPa*s and 200 mPa*s, between 4 mPa*s and 100 mPa*s, between 6 mPa*s and 80 mPa*s, between 10 mPa*s and 80 mPa*s or between 10 mPa*s and 50 mPa*s.

Said kit, wherein said viscosity between 1.7 mPa*s and 1500 mPa*s, between 2 mPa*s and 1400 mPa*s, between 2 mPa*s and 1000 mPa*s, between 2 mPa*s and 500 mPa*s, between 4 mPa*s and 1000 mPa*s, between 4 mPa*s and 500 mPa*s, between 4 mPa*s and 200 mPa*s, between 4 mPa*s and 100 mPa*s, between 6 mPa*s and 80 mPa*s, between 10 mPa*s and 80 mPa*s or between 10 mPa*s and 50 mPa*s of said differentiation medium is achieved by the presence of a viscosity enhancer in said differentiation medium, therefore said differentiation medium may comprise

i) a basal medium for animal or human cells, and

ii) a viscosity enhancer,

wherein said differentiation medium has a viscosity between 1.7 mPa*s and 1500 mPa*s, between 2 mPa*s and 1400 mPa*s, between 2 mPa*s and 1000 mPa*s, between 2 mPa*s and 500 mPa*s, between 4 mPa*s and 1000 mPa*s, between 4 mPa*s and 500 mPa*s, between 4 mPa*s and 200 mPa*s, between 4 mPa*s and 100 mPa*s, between 6 mPa*s and 80 mPa*s, between 10 mPa*s and 80 mPa*s or between 10 mPa*s and 50 mPa*s.

Said kit, wherein said viscosity enhancer may be biocompatible for the cells of said differentiation medium.

Said viscosity enhancer may be for example a non-gelling, biocompatible rheology modifier.

Rheology modifiers may be carrageenans, xanthan gum, and cellulose ether derivates such as methyl cellulose, carboxymethyl cellulose, and hydroxy ethyl cellulose, and mixtures thereof.

Said viscosity enhancer may be selected for example from the group of biocompatible rheology modifiers consisting of carrageenans, xanthan gum, and cellulose ether derivates such as methyl cellulose, carboxymethyl cellulose, and hydroxy ethyl cellulose, and mixtures thereof.

Preferentially said viscosity enhancer is methyl cellulose, carboxymethyl cellulose, hydroxy ethyl cellulose, or a combination thereof.

Said viscosity enhancer, wherein said viscosity enhancer is methyl cellulose, carboxymethyl cellulose or hydroxy ethyl cellulose, and wherein the concentration of methyl cellulose, carboxymethyl cellulose, or hydroxy ethyl cellulose is between 0.1% and 2% methyl cellulose, carboxymethyl cellulose, or hydroxy ethyl cellulose in said medium.

Said viscosity enhancer, preferentially methyl cellulose, carboxymethyl cellulose, or hydroxy ethyl cellulose, wherein said viscosity enhancer increases the viscosity of said differentiation medium to a value between 1.7 mPa*s and 1500 mPa*s.

Said kit, wherein said differentiation medium may comprise additionally one or more differentiation factors.

Said kit, wherein said one or more differentiation factors may be differentiation factors for differentiation of said multicellular aggregation to artificial neural tissue, to artificial cardiac tissue, to artificial kidney tissue or artificial retinal tissue.

Said kit, wherein said differentiation medium is for differentiation to artificial neural tissue, and wherein said differentiation medium optionally may comprise one or more differentiation factors selected from the group consisting of activator of Wnt signaling and an inhibitor for TGF-beta, activin and nodal signaling pathway.

Said kit, wherein said differentiation medium is for differentiation to artificial neural tissue, the kit may comprise

a) a differentiation medium comprising a basal medium for animal or human cells, wherein said differentiation medium has a viscosity between 1.77 mPa*s and 1496.82 mPa*s, between 2 mPa*s and 1400 mPa*s, between 2 mPa*s and 1000 mPa*s, between 2 mPa*s and 500 mPa*s, between 4 mPa*s and 1000 mPa*s, between 4 mPa*s and 500 mPa*s, between 4 mPa*s and 200 mPa*s, between 4 mPa*s and 100 mPa*s, between 6 mPa*s and 80 mPa*s, between 10 mPa*s and 80 mPa*s or between 10 mPa*s and 50 mPa*s,

b) a medium for generation of multicellular aggregation from human pluripotent stem cells comprising

i) a basal medium for animal or human cells

ii) a Rock inhibitor

c) a neural induction medium comprising

i) a basal medium for animal or human cells

ii) an inhibitor for TGF-beta, Activin and Nodal signaling pathway

iii) a Bone Morphogenetic Protein (BMP) inhibitor.

Said kit may further comprise

d) a medium for culturing the expanded multicellular aggregation according to the method as disclosed herein comprising

i) a basal medium for animal or human cells

ii) retinoic acid and retinol.

Said differentiation medium of said kit optionally may comprise one or more differentiation factors selected from the group consisting of activator of Wnt signaling and an inhibitor for TGF-beta, activin and nodal signaling pathway.

All definitions, characteristics and embodiments defined herein with regard to an aspect of the invention, e.g. the first aspect of the invention, also apply mutatis mutandis in the context of the other aspects of the invention as disclosed herein.

### Embodiments

In an embodiment of the invention, a differentiation medium as disclosed herein comprises a basal medium for animal or human cells and 0.5% methyl cellulose as a viscosity enhancer leading to a viscosity of said medium of about 10 to 15 mPA*sec, an activator of Wnt signaling and an inhibitor for TGF-beta, activin and nodal signaling pathway.

Pluripotent stem cells such as human induced pluripotent stem cells (iPSC) may be developed to a multicellular aggregation in medium for generation of multicellular aggregation from human pluripotent stem cells within 24 h. Said medium for generation of multicellular aggregation from human pluripotent stem cells may comprise a) a basal medium for animal or human cells, and ii) a Rock inhibitor.

Said multicellular aggregation may be cultured in a neural induction medium and may differentiate to artificial neural tissue within 4 days. The neural induction medium may comprise i) a basal medium for animal or human cells, ii) an inhibitor for TGF-beta, Activin and Nodal signaling pathway, and iii) a Bone Morphogenetic Protein (BMP) inhibitor.

Then the differentiated multicellular aggregation is cultured in suspension in above-mentioned differentiation medium for about 10 days for differentiation of the artificial neural tissue.

Optionally these artificial neural tissues may be cultured further by culturing said expanded multicellular aggregation in suspension culture in a medium for culturing the expanded multicellular aggregation comprising i) a basal medium for animal or human cells, and ii) retinoic acid and retinol for 10 to 15 days.

In one embodiment of the present invention the in vitro method for obtaining a brain organoid as disclosed herein comprises the additional step of investigating a developmental neurological tissue effect comprising decreasing or increasing the expression in a gene of interest in a cell at any stage during said method.

In one embodiment of the present invention the in vitro method for obtaining a brain organoid as disclosed herein comprises the additional step of screening a candidate therapeutic agent suitable for treating a developmental neurological tissue defect of interest, comprising performing said method of investigating a developmental neurological tissue effect as and administering the candidate agent to said cells at any stage during the method, preferably at all stages.

In one embodiment of the invention the brain organoid as disclosed herein is used in an in vitro method of testing a candidate drug for neurological effects, comprising administering a candidate drug to said organoid and determining an activity of interest of the cells of said organoid and comparing said activity to an activity of cells to the organoid without administering said candidate drug, wherein a differential activity indicates a neurological effect.

In one embodiment of the invention the brain organoid as disclosed herein is used in an in-vitro method of obtaining a differentiated neural cell comprising the step of providing said organoid and isolating a differentiated neural cell of interest, or comprising the step of generating said organoid according to the method for obtaining a brain organoid as disclosed herein further comprising the step of isolating a differentiated neural cell of interest.

### Definitions

Unless defined otherwise, technical and scientific terms used herein have the same meaning as commonly understood by one of ordinary skill in the art to which this invention belongs. The term “pluripotent stem cell” as used herein refers to cells being capable to self-renew and have the potential to differentiate into any of the embryonic germ layers endoderm, mesoderm and ectoderm and cells derived from this. These criteria hold true for embryonic stem cells (ESC) and induced pluripotent stem cells (iPSC). Normally, these cells are of human origin, i.e. human cells. Different degrees of pluripotency are known in the art, referred to as “primed state” pluripotent stem cells, “naive state” pluripotent stem cells or “reset stage” pluripotent stem cells.

The term embryonic stem cells (ESCs) as used herein refers to human pluripotent stem cells derived from the inner cell mass of a blastocyst at an early-stage before implantation. ESCs are capable to self-renew and have the potential to differentiate into any of the embryonic germ layers endoderm, mesoderm and ectoderm and cells derived from this. ESCs show expression of the pluripotency marker OCT3/4. Human embryonic stem cells can be isolated from embryos without destruction as disclosed e.g. in WO 03/046141.

The term “induced pluripotent stem cells (iPSC)” as used herein refers to human pluripotent cells generated by conversion of cells of lower potency, i.e. more differentiated cells, typically a somatic cell, to a state of pluripotency, the resulting cells being capable to self-renew and having the potential to differentiate into any of the embryonic germ layers endoderm, mesoderm and ectoderm and cells derived from this. iPSCs show expression of the pluripotency marker OCT3/4. Reprogramming may be achieved by methods known in the art such as nuclear transfer, cell fusion, or factor induced reprogramming, i.e. induced expression of one or more reprogramming factors, such as but not limited to OCT3/4, SOX2, KLF4, C-MYC, NANOG, LIN28, etc. Reprogramming factors may be introduced as nucleic acids, or proteins by viral transduction or by transfection. Different culture conditions and reprogramming factor combinations may result in different degrees of pluripotency, referred to as “primed state” pluripotent stem cells, “naive state” pluripotent stem cells or “reset stage” pluripotent stem cells.

The terms “artificial tissue structure” or “organoid” may be used interchangeably, these terms as used herein refer to a network of cells that has been developed from pluripotent stem cells resembling in morphology and/or physiology a human tissue. The cellular network recapitulates cellular structures/tissue architectures seen in processes of human organ development. Due to that an artificial tissue structure can show similarities to different developmental stages, depending on the time point of analysis. Depending on the developing organ different tissue architectures are expected. As the artificial tissue structures are developed in-vitro and are not identical to naturally (in-vivo) grown tissue structures that develop during e.g. embryogenesis they are “artificial”.

The terms “artificial neural tissue” or “brain organoid” may be used interchangeably, these terms as used herein refer to a multicellular structure resembling the morphology of developing human brain parts. These multicellular structures show the expression of typical neural markers, observed during human brain development. Moreover the overall tissue architecture, cell types, cell localization and cell complexity is comparable with the developing human brain. Contrary to that brain spheroids show less complex structures and lack tissue complexity. For that reason they are not part of the definition for a brain organoid. As the artificial neural tissue is developed in vitro and is not identical to naturally (in vivo) grown neural tissue that develop during e.g. embryogenesis it is “artificial”.

The term “a multicellular aggregation derived from pluripotent stem cells” as used herein refers to an aggregate of cells comprising pluripotent stem cells that emerges when pluripotent stem cells are cultured in a pluripotent stem cell medium such as the “medium for generation of multicellular aggregation from pluripotent stem cells comprising” as disclosed herein. Said multicellular aggregation may also be termed “embryoid body”, a further standard term in the prior art. The multicellular aggregation may be developed further to a more specialized artificial tissue structure or specialized tissue.

The term multicellular aggregation as used herein defines an assembly of several cells in one three dimensional structures. Cells within a multicellular aggregation might be of the same kind e.g. pluripotent stem cells or of different differential stages, depending on the time point of differentiation.

A three-dimensional matrix is a three-dimensional structure of a biocompatible matrix such as an extracellular matrix.

The term “extracellular matrix” (ECM) as used herein refers to a collection of extracellular molecules secreted by connective tissue that provides structural and biochemical support to the surrounding cells (naturally occurring ECM) and/or refers to natural, semi-synthetic and synthetic biomaterials or mixtures thereof that can build matrices or scaffolds that mimic a cellular niche e.g. for stem cells during culturing them. All these structural supports, matrices and scaffolds have the inherent feature that cells such as pluripotent stem cells can attach to these structures, i.e. to the ECM a three-dimensional matrix), and therefore said cells are not in suspension in a cell culture medium.

A scaffold provides a three-dimensional network. Suitable synthetic materials for said scaffold comprise polymers selected from porous solids, nanofibers, and hydrogels such as, for example, peptides including self-assembling peptides, hydrogels composed of polyethylene glycol phosphate, polyethylene glycol fumarate, polyacrylamide,

polyhydroxyethyl methacrylate, polycellulose acetate, and/or co-polymers thereof. ECM is composed of a variety of polysaccharides, water, elastin, and glycoproteins, wherein the glycoproteins comprise collagen, entactin (nidogen), fibronectin, and laminin. ECM is secreted by connective tissue cells. Different types of ECM are known, comprising different compositions including different types of glycoproteins and/or different combination of glycoproteins. Said ECM can be provided by culturing ECM-producing cells, such as for example fibroblast cells, in a receptacle, prior to the removal of these cells and the addition of e.g. pluripotent stem cells. Examples of extracellular matrix-producing

cells are chondrocytes, producing mainly collagen and proteoglycans, fibroblast cells, producing mainly type IV collagen, laminin, interstitial procollagens, and fibronectin, and colonic myofibroblasts producing mainly collagens (type I, III, and V), chondroitin sulfate proteoglycan, hyaluronic acid, fibronectin, and tenascin-C. Alternatively, said ECM is commercially provided. Examples of commercially available extracellular matrices are extracellular matrix proteins (Invitrogen) and Matrigel™ (BD Biosciences).

Again, the ECM has a solidified structure that allows for attachment/adhesion of cells in culture. Cell culture is the process by which cells are grown under controlled conditions (also termed “culturing”), generally outside their natural environment. After the cells of interest have been isolated e.g. from living tissue, they can subsequently be maintained under carefully controlled conditions. These conditions vary for each cell type, but generally consist of a suitable vessel with a substrate or medium that supplies the essential nutrients (amino acids, carbohydrates, vitamins, minerals), growth factors, hormones, and gases (CO2, O2), and regulates the physio-chemical environment (pH buffer, osmotic pressure, temperature). Most cells require a surface or an artificial substrate (adherent or monolayer culture) whereas others can be grown free floating in culture medium (suspension culture, or “in suspension”). Therefore, the term “suspension (cell) culture” means that the cells or multicellular units or multicellular aggregates of a culture grow free floating in the culture medium, i.e. they are in suspension.

A “multicellular aggregation derived from human pluripotent stem cells that has been induced to differentiate to an artificial tissue structure” means for example in case of artificial neural tissue, during the development, the multicellular cell aggregates form polarized neuroepithelial structures and a neuroepithelial sheet, which will develop several round clusters (rosettes). These steps may be controlled by neural induction medium as disclosed herein and e.g. described by Eiraku (2008), US 2011/0091869 A1 and WO 2011/055855 A1

The term “differentiation medium for differentiation and expansion of a multicellular aggregation in suspension derived from human pluripotent stem cells that has been induced to differentiate to an artificial tissue structure” as used herein means a further differentiation and/or development of said multicellular aggregation to an artificial tissue structure, i.e. a more differentiated cellular structure than said multicellular aggregation. Therefore, alternatively the term “differentiation medium for further differentiation and expansion of a multicellular aggregation in suspension derived from human pluripotent stem cells that has been induced to differentiate to an artificial tissue structure” may be used herein.

The term “differentiation and expanding of a multicellular aggregation that has been induced to differentiate to an artificial tissue structure” means for example in case artificial neural tissue, the polarized neuroepithelial structures and a neuroepithelial sheet, which will develop several round clusters (rosettes) will develop further to more differentiated structures.

The term “basal medium for animal or human cells” as used herein refers to a defined synthetic medium for animal or human cells that is buffered preferably at a pH between 7. 2 and 7.6, preferentially at about a pH of 7.4 with a carbonate-based buffer, while the cells are cultured in an atmosphere comprising between 5% and 10% CO2, preferably about 5% CO2. A preferred basal medium suited for animal or human cells may be selected from DMEM/F12 and RPMI 1640 supplemented with glutamine, insulin, Penicillin/streptomycin and transferrin. In a further preferred embodiment, Advanced DMEM/F12 or Advanced RPMI is used, which is optimized for serum free culture and already includes insulin. In this case, said Advanced DMEM/F12 or Advanced RPMI medium is preferably supplemented with glutamine and Penicillin/streptomycin. It is furthermore preferred that said medium is supplemented with a purified, natural, semi-synthetic and/or synthetic growth factor and does not comprise an undefined component such as fetal bovine serum or fetal calf serum. Supplements such as, for example, B27, N-Acetylcysteine and N2 stimulate proliferation of some cells and can further be added to the medium, if required.

The viscosity of a fluid is the measure of its resistance to gradual deformation by shear stress. For liquids such as cell media, it corresponds to the informal concept of “thickness”.

One way for measuring kinematic viscosity is the glass capillary viscometer. Another option may be the calculation from x gram or % of viscosity enhancer in solution to viscosity (Pa*s) by using the following formula

η^{1/8}=(c·α)+1

η=(c·α+1)⁸

η=solution viscosity in mPa*s

α=constant specific for each methyl cellulose

c=concentration of methyl cellulose in solution in %

Example 0.5% Methyl cellulose; α=0.747

η=(0.5%·0.474+1)⁸

η=12.66 mPa·s

The physical unit of viscosity is pascal second (Pa*s). mPa*s means milli-pascal second. The range of viscosity that can be used in the differentiation medium as disclosed herein was exemplary determined by using the viscosity enhancer methyl cellulose. A viscosity of said medium of 1.7 mPa*s correlates to 0.1% methyl cellulose in said medium, a viscosity of said medium of 3.9 mPa*s correlates to 0.25% methyl cellulose in said medium, a viscosity of said medium of 12.66 mPa*s correlates to 0.5% methyl cellulose in said medium, a viscosity of said medium of 86.76 mPa*s correlates to 1% methyl cellulose in said medium, a viscosity of said medium of 1500 mPa*s correlates to 2% methyl cellulose in said medium.

The term “viscosity enhancer” may be any substance that can increase the viscosity of a liquid such as a medium to a value between 1.7 mPa*s and 1500 mPa*s, between 2 mPa*s and 1400 mPa*s, between 2 mPa*s and 1000 mPa*s, between 2 mPa*s and 500 mPa*s, between 4 mPa*s and 1000 mPa*s, between 4 mPa*s and 500 mPa*s, between 4 mPa*s and 200 mPa*s, between 4 mPa*s and 100 mPa*s, between 6 mPa*s and 80 mPa*s, between 10 mPa*s and 80 mPa*s or between 10 mPa*s and 50 mPa*s and may be biocompatible to cells that are contained in such medium. The viscosity enhancer may be for example selected from the group consisting of non-gelling, biocompatible rheology modifiers such as carrageenans, xanthan gum, cellulose ether derivates such as methyl cellulose, carboxymethyl cellulose, and hydroxy ethyl cellulose, and mixtures thereof.

It is a feature of the viscosity enhancer that it does not build a three-dimensional matrix in the liquid such as a cell culture medium.

The viscosity enhancer may be cellulose ether derivates selected from the group consisting of methyl cellulose, carboxymethyl cellulose, and hydroxy ethyl cellulose, and mixtures thereof. In a preferred embodiment of the invention, the viscosity enhancer may be methyl cellulose.

Rheology modifiers (thickeners) as used herein affect the stability and flow properties of a liquid such as a cell culture medium. They should be non-gelling, i.e. they should not form a gel. They also should be biocompatible. Examples may be carrageenans, xanthan gum, and cellulose ether derivates such as methyl cellulose, carboxymethyl cellulose, and hydroxy ethyl cellulose, and mixtures thereof.

Preferentially said viscosity enhancer may be methyl cellulose, carboxymethyl cellulose, hydroxy ethyl cellulose, or a combination thereof.

Said viscosity enhancer, wherein said viscosity enhancer may be methyl cellulose, carboxymethyl cellulose or hydroxy ethyl cellulose, and wherein the concentration of methyl cellulose, carboxymethyl cellulose, or hydroxy ethyl cellulose may be between 0.1% and 2% methyl cellulose, carboxymethyl cellulose, or hydroxy ethyl cellulose in said medium.

Said viscosity enhancer, preferentially methyl cellulose, carboxymethyl cellulose, hydroxy ethyl cellulose, wherein said viscosity enhancer increases the viscosity of said differentiation medium to a value between 1.7 mPa*s and 1500 mPa*s.

Carragenans (or carragenins) are a family of linear sulfated polysaccharides that are extracted from red edible seaweeds. There are three main varieties of carrageenan, which differ in their degree of sulfation. Kappa-carrageenan has one sulfate group per disaccharide, iota-carrageenan has two, and lambda-carrageenan has three.

Xanthan gum is a polysaccharide with many industrial uses. It is an effective thickening agent and stabilizer to prevent ingredients from separating. It can be produced from simple sugars using a fermentation process, and derives its name from the species of bacteria used, Xanthomonas campestris.

The term biocompatible in the context of a biocompatible material/substance means that the material/substance is inert and/or non-toxic to cells, e.g. of a cell culture or of a human body.

The term “differentiation factor” or “differentiation agent” as used herein refers to an agent that triggers and/or induces differentiation or further differentiation from a less specified cell or tissue to a more specified cell or tissue.

“Inhibitor” as used herein, refers to a compound or molecule (e.g., small molecule, peptide, peptidomimetic, natural compound, siRNA, anti-sense nucleic acid, aptamer, or antibody) that interferes with (e.g., reduces, decreases, suppresses, eliminates, or blocks) the signaling function of the molecule or pathway. An inhibitor can be any compound or molecule that changes any activity of a named protein (signaling molecule, any molecule involved with the named signaling molecule, a named associated molecule

“Activators,” as used herein, refer to compounds that increase, induce, stimulate, activate, facilitate, or enhance activation the signaling function of the molecule or pathway, e.g., Wnt signaling,

As used herein, the term “differentiation” refers to a process whereby an unspecialized cell such as a pluripotent stem cell acquires the features of a specialized cell such as a neuron, heart, liver, or muscle cell. Differentiation is controlled by the interaction of a cell's genes with the physical and chemical conditions outside the cell, usually through signaling pathways involving proteins embedded in the cell surface.

As used herein, the term “inducing differentiation” in reference to a cell refers to changing the default cell type (genotype and/or phenotype) to a non-default cell type (genotype and/or phenotype). Thus, “inducing differentiation in a (human) pluripotent stem cell” refers to inducing the pluripotent stem cell to divide into progeny cells with characteristics that are different from the pluripotent stem cell, such as genotype (e.g., change in gene expression) and/or phenotype (e.g., change in expression of a protein marker).

The Wnt signaling pathway is defined by a series of events that occur when a Wnt protein binds to a cell-surface receptor of a Frizzled receptor family member. This results in the activation of Disheveled family proteins which inhibit a complex of proteins that includes axin, GSK-3, and the protein APC to degrade intracellular β-catenin. The resulting enriched nuclear β-catenin enhances transcription by TCF/LEF family transcription factors.

A Wnt agonist (or Wnt activator) is defined herein as an agent that activates TCF/LEF-mediated transcription in a cell. Wnt agonists are therefore selected from true Wnt agonists that bind and activate a Frizzled receptor family member including any and all of the Wnt family proteins, an inhibitor of intracellular β-catenin degradation, and activators of TCF/LEF.

Said Wnt agonist may be selected from the group consisting of Wnt family member, R-spondin family, Norrin, and an GSK-inhibitor.

The Wnt family member includes Wnt-1/Int-1; Wnt-2/Irp (Int-1-related Protein); Wnt-2b/13; Wnt-3/Int-4; Wnt-3a; Wnt-4; Wnt-5a; Wnt-5b; Wnt-6; Wnt-7a; Wnt-7b; Wnt-8a/8d; Wnt-8b; Wnt-9a/14; Wnt-9b/14b/15; Wnt-10a; Wnt-10b/12; Wnt-11; and Wnt-16.

The R-spondin family comprises R-spondin-1, R-spondin-2, R-spondin-3, and R-spondin-4. Known GSK-inhibitors comprise small-interfering RNAs (siRNA), lithium, kenpaullone, SB 216763 and SB 415286 (Sigma-Aldrich), and FRAT-family members and FRAT-derived peptides that prevent interaction of GSK-3 with axin.

In an embodiment of the invention, said Wnt agonist comprises or consists of R-spondin 1. R-spondin 1 may be preferably added to the cell culture medium at a concentration of at least 50 ng/ml, more preferred at least 100 ng/ml, more preferred at least 200 ng/ml, more preferred at least 300 ng/ml, more preferred at least 500 ng/ml. A most preferred concentration of R-spondin 1 is approximately 500 ng/ml or 500 ng/ml. During culturing of stem cells, said Wnt family member is preferably added to the cell culture medium every second day, while the culture medium is refreshed preferably every fourth day.

In another embodiment of the invention, a Wnt agonist is selected from the group consisting of: R-spondin, Wnt-3a and Wnt-6. More preferably, R-spondin and Wnt-3a are both used as Wnt agonist. Preferred concentrations may be approximately 500 ng/ml or 500 ng/ml for Rspondin and approximately 100 ng/ml or 100 ng/ml for Wnt3a.

Inhibitors for TGF-beta, activin and nodal signaling pathway are substances either naturally occurring cytokines or chemically synthesized small molecules that prevent the activation of the signaling cascade (of a specific pathway). Downstream cascades will not become activated and therefore the activation or inhibition of downstream genes is prevented. Signaling pathway inhibitors might act on different levels of the pathway e.g. signaling receptor, key regulating e.g. enzymes.

An organoid is a miniaturized and simplified version of an organ produced in vitro in three dimensions that shows realistic micro-anatomy. They are derived from one or a few cells from a tissue, embryonic stem cells or induced pluripotent stem cells, which can self-organize in three-dimensional culture owing to their self-renewal and differentiation capacities.

A brain organoid is a miniaturized and simplified version of a brain produced in vitro in three dimensions that shows realistic micro-anatomy of a brain. Structures of such organoid are described e.g. herein. A specific variant of a brain organoid is a cerebral organoid.

This invention is further illustrated by the following examples, which are not to be construed in any way as imposing limitations upon the scope thereof.

### EXAMPLES

### Example 1: Generation of PSC Derived Cerebral Organoids Using a Medium with Viscosity Enhancer

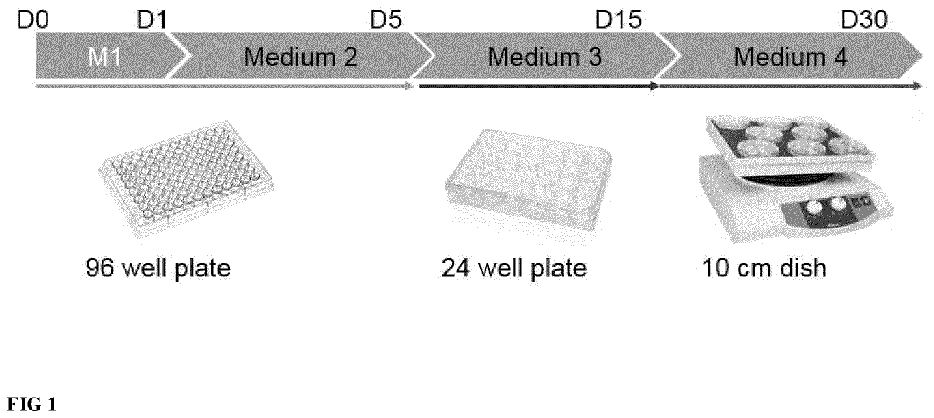

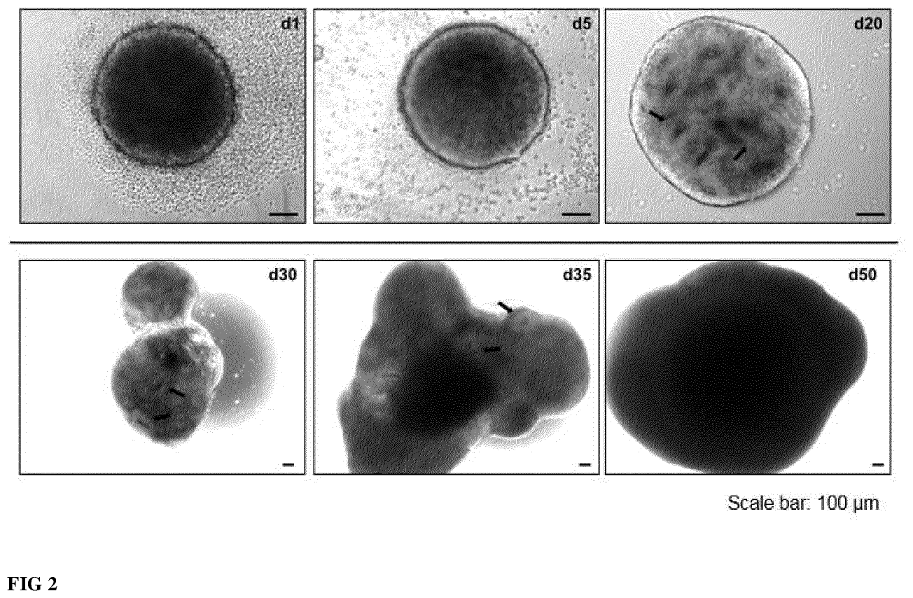

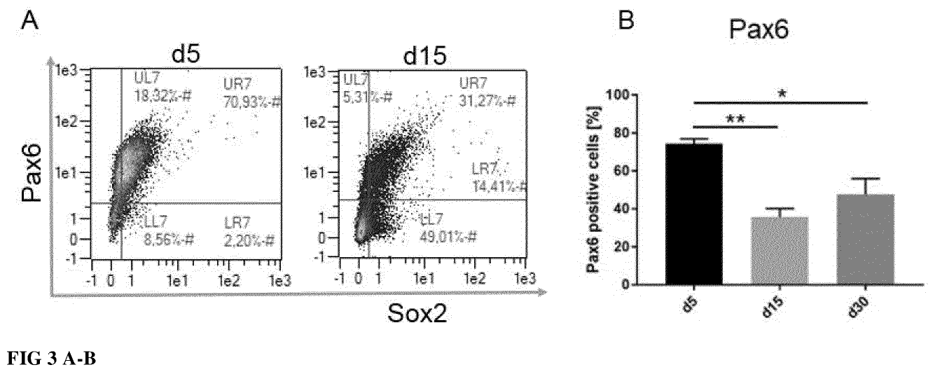

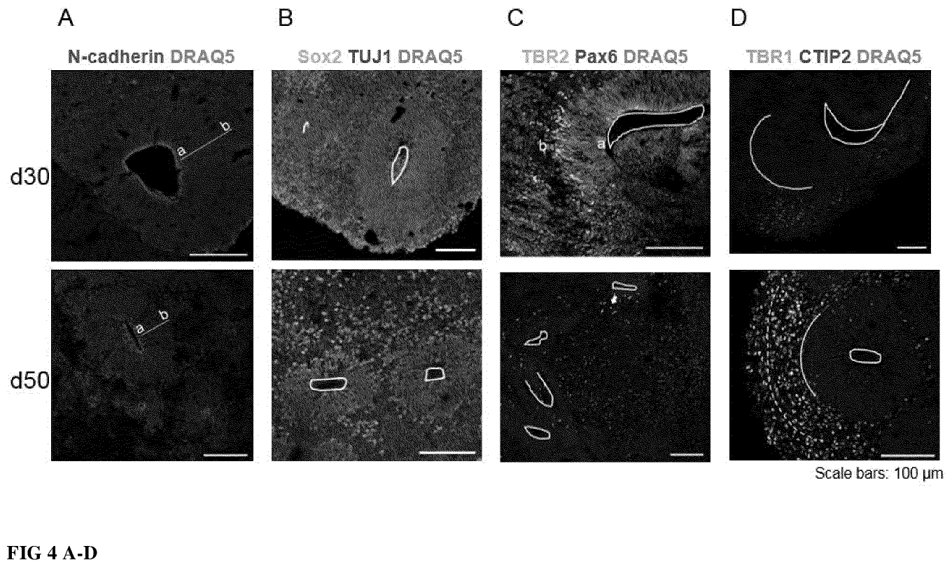

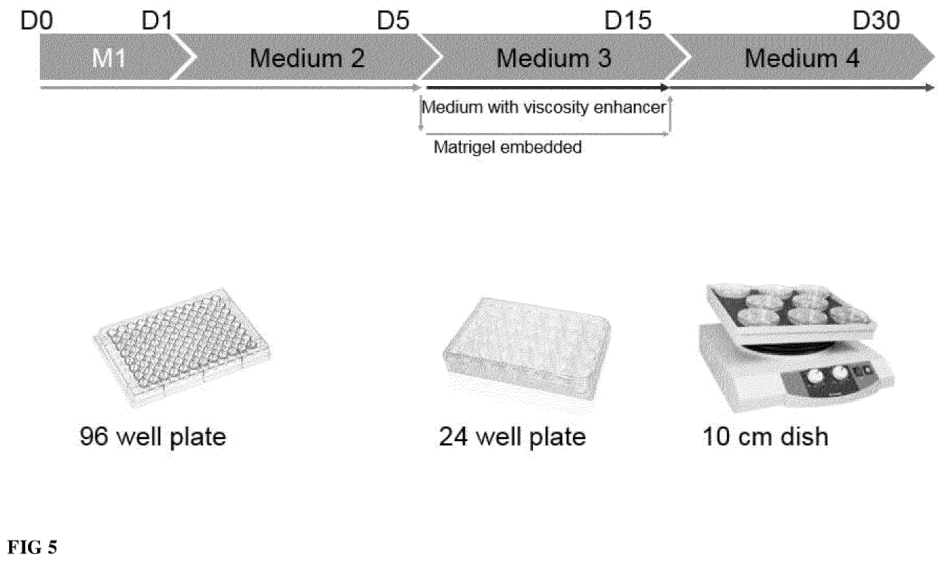

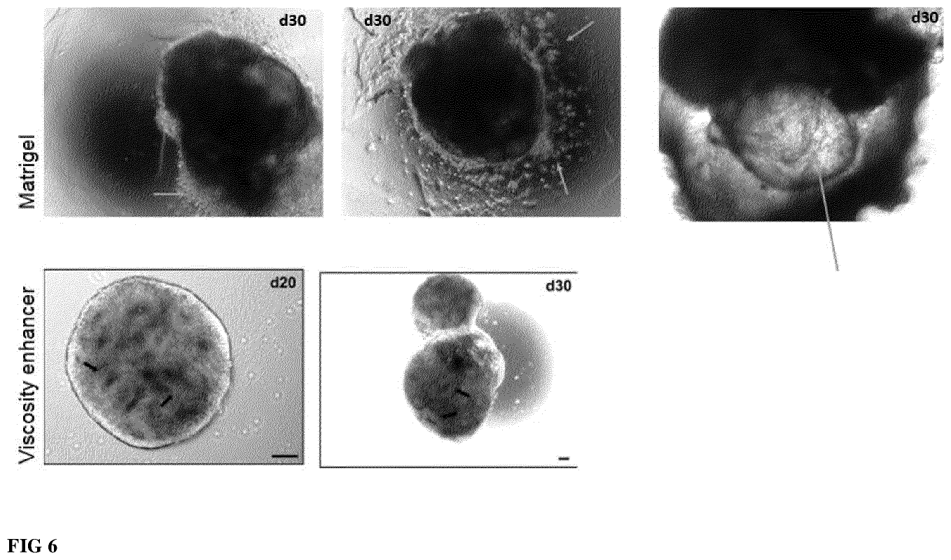

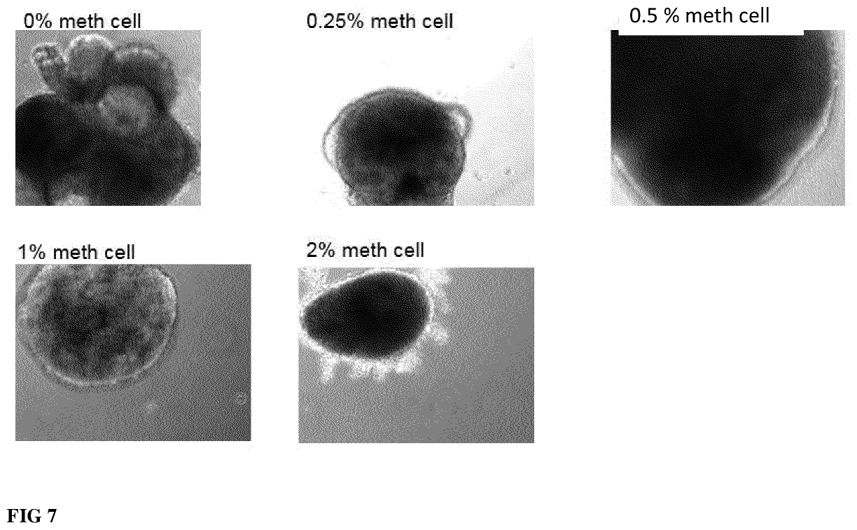

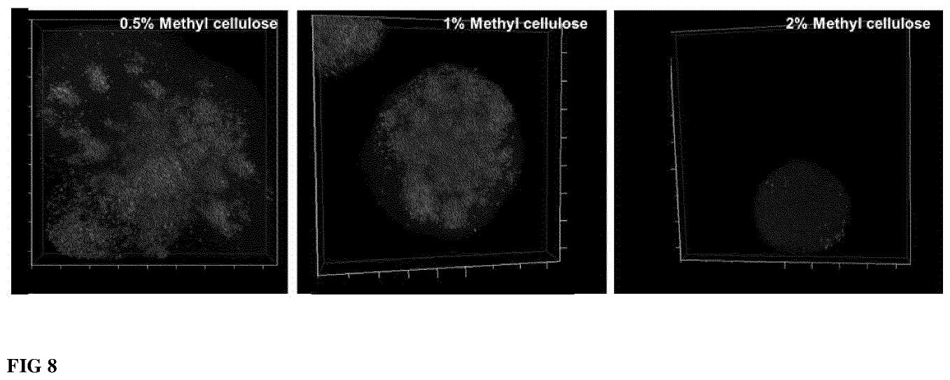

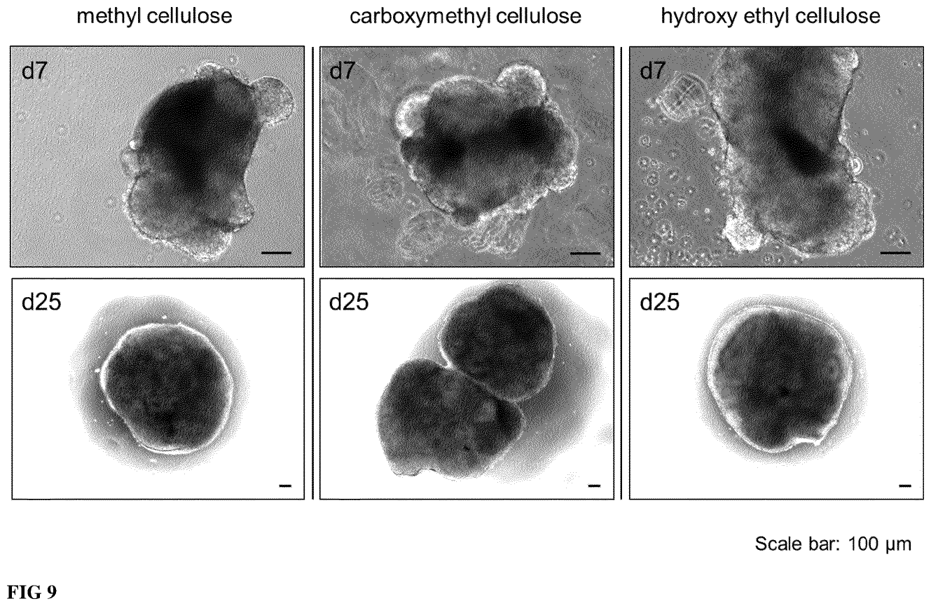

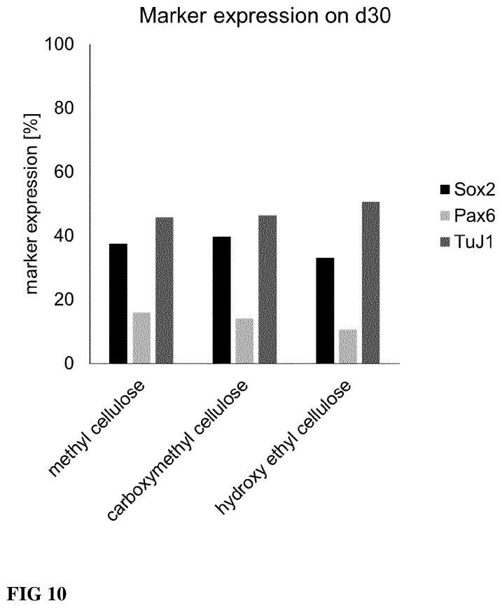

For the generation of human brain organoids human pluripotent stem cells were dissociated into single cells using standard procedures. Depending on the stem cell clone 7500-20000 cells were seeded into 96 well ultra-low attachment plates in standard stem cell medium lacking typical cytokines such as activin A, bFGF or TGF beta. Within 24 h cells clustered and the formation of round dense structures was observed. Roughly 24 h after seeding the medium was replaced by neural induction media such as shown in Quian et al (2016, Cell:165: 1238; 2018, Nature Protocols, 13:565) and in WO2014090993A1 (neural induction medium) (FIG. 1). Media exchanges were done every other day until day five. On day 5 early neural tissues were transferred to 24 well plates and medium 3 containing 0.5% methyl cellulose as containing the viscosity enhancer as disclosed herein was added. On day 15 the developing neural tissue was transferred to 10 cm dishes, which are placed onto a shaker (FIG. 1). Depending on the desired developmental stage the organoids could be cultivated >100 days. From day 15 organoids were cultured in cerebral organoid differentiation medium such as described in Quian et al (2016, Cell:165: 1238; 2018, Nature Protocols, 13:565) and in WO2014090993A1.

During the generation of the organoid structure a several morphological changes could be observed using transmitted light microscopy (FIG. 2). 24 h after seeding round multicellular aggregates formed. These structures showed an integrated border and a dense core, which was surrounded by a more transparent ring. Until day 5 the overall size of the multicellular structure increased (FIG. 2 d5). Moreover the inner core of the organoid showed a more heterogenous structure, indicating structural rearrangements in the inner of the organoid. The structure was still dense and compact. As development proceeds the organoids grow in size and some structural rearrangements could be observed. On day 20 and 30 round structures in the inner of the organoids developed. Typically these structures showed an inner ring that surrounds a “hollow” black cavity. The inner ring was further surrounded by an outer ring, showing the edges of the structure. This arrangement is similar to the embryonic brain development, where the fluid filled ventricle is lined by a progenitor zone that has a dominant apical membrane near to the ventricle and a basal membrane on the basal site. These structures morphologically resemble the neural progenitor zones. As the organoids grew older the structure became more dense. No inner structures could be detected by transmitted light microscopy.

### Example 2: Characterization of Cerebral Organoids Using Flow Cytometry

The change in neural marker expression during organoid development was measured over the time of development in order to assess the degree of neural induction. The organoids were analyzed on day 5, 15 and day 30 of differentiation. To that end organoids were harvested using the Multi Tissue Dissociation Kit 3™ (Miltenyi Biotec GmbH) in order to obtain single cells. In short: The organoids were transferred into an Eppendorf cup, washed twice with dPBS and then the enzyme mix was added. Depending on the developmental stage the cerebral organoids were incubated for 10 minutes @ 37° C. (day 5/Day 15 organoids) or 15 minutes (day 30 organoids). Afterwards a stopping reagent was added and organoids were dissociated by pipetting up and down. The single cell suspension was stained for the expression of the neural progenitor markers Pax6 and Sox2 using the FoxP3 Staining Buffer Set (Miltenyi Biotec GmbH). Stained cells were analyzed using the MACS Quant Analyzer and MACS Quantify Software.

On day 5 high expression of the neural progenitor markers Pax6 and Sox2 could be observed (FIG. 3). The Dot pots show an overlapping expression of >90%, indicating a high neural induction of the stem cells and the presence of neural progenitor cells. In contrast to that on day 15 and 30 the expression of both markers was decreased to ˜35%, indicating a decrease in the neural progenitor population. This is in line with the processes taking place during neural development, because neural progenitor cells deplete over time and generate neurons, thus explaining the decrease in progenitor population.

### Example 3: Characterization of Organoids

Cerebral organoids were generated as described in Example 1. The characterization of the brain organoids was performed on day 30 and day 50. To that end organoids were fixed, cryo-sectioned (20 μM) and stained with specific antibodies that are typical for neural development. The complete protocol is described WO2014090993A1.

Representative cross sections are shown in FIG. 4. In order to show the integrity of the apical membrane, the organoids were stained for the expression of N-Catherin. High expression was observed surround the ventricles, showing the presence of an apical membrane. The expression was independent of the analysis time point.

Moreover the organoid were analyzed for the expression of the neural progenitor marker Sox2 The expression of Sox2 was mainly observed near to the ventricles, representing neural progenitor layers, which are expected during neural development.

The neural progenitor layer is surrounded by TuJ1 positive cell layers. This marker is expressed in early neurons, which confirms the early neural output in the organoids. The arrangement of progenitor markers at the ventricle and a surrounding TuJ1 staining correlates with standard neural developmental processes. This arrangement is also observed on day 50.

Further markers known for neural development are TBR2 and Pax6. Pax 6 labels neural progenitor cells that are localized near to the ventricle. The expression of Sox2 and Pax 6 overlaps. Both markers label cells localized near to the ventricle. TBR-2 positive cells represent a different neural progenitor population which is positioned more basally, making up a subventricular zone.

Furthermore the expression of the cortical plate marker TBR1 and the deep layer neurons was analyzed. On day 50 both markers can be detected basally to the ventricular zone. As observed in neural development TBR1 is found at the very basal site representing the developing cortical plate. In contrast to that CTIP2 is found apically of the cortical plate, representing the formation of deep layer neurons.

At the end we can say that all characteristic markers for organoids are expressed.

### Example 4: Comparison Differentiation Medium with Methyl Cellulose as Viscosity Enhancer and Matrigel™ Embedding

To compare the methyl cellulose media condition with Matrigel™ embedded organoids, two different protocols were used. Organoids of the methyl cellulose media condition were generated using the protocol explained in example 1. In contrast to that the protocol was adapted for the Matrigel™ condition organoids. In this condition the neural tissue was embedded into a Matrigel™ droplet on day 5. The embedding steps are described Lancaster et al.; Nature Protocols volume 9, pages 2329-2340 (2014). No medium with viscosity enhancer was used in this condition (FIG. 5). All other steps were the same as in example 1. Comparing both conditions by transmitted light microscopy with each other some differences became visible (FIG. 6). A dense structure could be observed for Matrigel™ embedded organoids. Moreover some neural outgrowth indicated by arrows can be shown. Some cells seem to migrate into the Matrigel™. No smooth surface can be observed.

In contrast to that organoids generated without Matrigel™ but using the methyl cellulose medium showed a smooth surface without any neural outgrowth. Moreover organoids in the Matrigel™ condition showed a tendency towards formation of unspecific structures containing fluid filled cavities (cyst like structures). These structures were missing, when using the viscosity enhancer.

Furthermore the amount of ventricle like structures/organoid was analyzed. In order to count the ventricle like structures organoids were stained with the neural progenitor marker Sox 2. Afterwards organoids were made transparent using the ECi tissue clearing protocol Klingberg et al.; JASN February 2017, 28 (2) 452-459. Fluorescent pictures were taken using confocal microscopy. Ventricle like zones were counted for Matrigel™ and viscosity enhancer conditions and 2 different iPS cell clones and presented in a diagram. For F10 and K10<5 ventricles were counted (FIG. 8). In contrast to that, an increased ventricle count was observed in organoids that were generated using the media as disclosed herein.

### Example 5: Titration of Different Media Viscosities Using Different Methyl Cellulose Concentrations

In order to determine the range of viscosity that support organoid formation, different methyl cellulose viscosities were tested. To that end the concentration of the viscosity enhancer was adjusted to 0%, 0.25%, 0.5%, 1% or 2%. All other steps in the protocol stayed the same. FIG. 7A shows transmitted light microscopy data obtained from organoids cultivated in 0%, 0.25%, 0.5%, 1% or 2% methyl cellulose. The cultivation of organoids without any viscosity enhancer leads to a dissolved structure of the organoids. They become less compact and more fringy. Over time the majority of these organoids dissolve completely, thus leading to highly decreased yields in organoids. In order to find out whether progenitor zones formed in the inner of the organoid, the organoids were stained for the proliferation marker Ki67 and cleared using the standard tissue clearing procedures based on ECi. The cleared organoids were analyzed using confocal microcopy and Z stacks were reconstructed to illustrate a complete organoid including the ventricle like zones (FIG. 7B). Moreover looking at the amount of ventricle like structures, no progenitor zones could be observed. Therefore the generation of organoids without a viscosity enhancer is not favorable.

Looking at the pictures generated for 0.25%-1% Methyl cellulose these organoids were more dense and show a very compact structures. Moreover they had an integrated border, and some cellular structures within the organoids can be observed. This indicates the successful generation of brain organoids containing typical progenitor zones. This can be further emphasized by tissue clearing data, where a high number of ventricle like structures could be observed.

Interestingly, after adding 2% methyl cellulose to the medium, the medium becomes highly viscous. The organoids show decreased sizes compared to other conditions. They are very compact and without any morphological structures inside. This indicates that the organoids might not form typical progenitor zones. Moreover no ventricle like structures could be detected after tissue clearing.

### Example 6: Comparison of Different Viscosity Enhancers

In order to evaluate availability of other viscosity enhances we compared morphology of organoids generated after addition of methylcellulose or carboxymethyl cellulose and hydroxy ethyl cellulose. To that end viscosity of medium 3 was enhanced using 0.5% methylcellulose, 0.21% carboxymethyl cellulose and 0.25% hydroxy ethyl cellulose. All other steps in the protocol stayed the same (Example 1). FIG. 9 shows transmitted light microscopy data obtained from organoids cultivated in 0.5% methylcellulose, 0.21% carboxymethyl cellulose or 0.25% hydroxy ethyl cellulose. On day 7 of differentiation organoids in all three conditions show similar morphologies. Some small structures can be observed in the inner parts and bulges at the surface, both indicating ongoing differentiation and proliferation. Until day 25 the size of the organoids increased and structural rearrangements could be observed. In all three conditions round structures in the inner of the organoids developed, morphologically resembling the ventricle, surrounded by neural progenitor zones.

Moreover the organoids were analyzed for the expression of the neural progenitor markers Sox2 and Pax6 and cytoskeletal marker in early neurons TuJ1 on day 30. Therefor flow cytometric measurement was performed as described in Example 2. FIG. 10 shows the marker expression at day 30 which is similar in all three conditions. TuJ1 expression is around 45-50%, whereas Sox2 and Pax6 expression is less strong, which is in line with the processes taking place during neural development. Neural progenitor cells deplete over time and generate neurons, thus explaining the decrease in progenitor population.

1) An in-vitro method for obtaining a brain organoid comprising
a) providing a multicellular aggregation of human pluripotent stem cells,
b) culturing said multicellular aggregation in a neural induction medium thereby inducing the multicellular aggregation to differentiate to a brain organoid,
c) culturing said differentiated multicellular aggregation in suspension in a differentiation medium, wherein said differentiation medium has a viscosity between 1.7 mPa*s and 1500 mPa*s, thereby expanding the cells in a multicellular aggregation, wherein said cells are able to differentiate further.

2) The method according to claim 1, wherein said differentiation medium comprises a viscosity enhancer that is biocompatible for the cells of said differentiation medium.

3) The method according to claim 2, wherein said viscosity enhancer does not build a three-dimensional matrix in the cell culture medium.

4) The method according to claim 2 or 3, wherein said viscosity enhancer is selected from the group consisting of non-gelling, biocompatible rheology modifiers such as carrageenans, xanthan gum, and cellulose ether derivates such as methyl cellulose, carboxymethyl cellulose, and hydroxy ethyl cellulose, and mixtures thereof.

5) The method according to claim 4, wherein said viscosity enhancer is methyl cellulose, carboxymethyl cellulose, or hydroxy ethyl cellulose.

6) The method according to claim 5, wherein the concentration of methyl cellulose, carboxymethyl cellulose, or hydroxy ethyl cellulose is between 0.1% and 2% methyl cellulose, carboxymethyl cellulose, or hydroxy ethyl cellulose in said medium.

7) The method according to any one of claims 1 to 6, wherein said differentiation medium comprises
i) said basal medium for animal or human cells, and
ii) said viscosity enhancer; and optionally
iii) an activator of Wnt signaling and/or an inhibitor for TGF-beta, activin and nodal signaling pathway.

8) The method according to any one of claims 1 to 7, wherein said method comprises the additional step:
d) culturing said expanded multicellular aggregation of cells from step c) in a suspension culture.

9) A brain organoid obtainable by a method according to any one of claims 1 to 8.

10) The use of a viscosity enhancer for adjusting the viscosity of a cell medium used for obtaining an artificial tissue structure derived from human pluripotent stem cells, wherein said viscosity is between 1.7 mPa*s and 1500 mPa*s.

11) The use according to claim 10, wherein said viscosity enhancer does not build a three-dimensional matrix in the cell culture medium.

12) The use of claim 11, wherein said viscosity enhancer is selected from the group consisting of carrageenans, xanthan gum, and cellulose ether derivates such as methyl cellulose, carboxymethyl cellulose, and hydroxy ethyl cellulose, and mixtures thereof.

13) The use of claim 12, wherein said viscosity enhancer is methyl cellulose, carboxymethyl cellulose, or hydroxy ethyl cellulose.

14) The use according to claim 13, wherein the concentration of methyl cellulose, carboxymethyl cellulose, or hydroxy ethyl cellulose is between 0.1% and 2% methyl cellulose, carboxymethyl cellulose, or hydroxy ethyl cellulose in said medium.

15) A kit comprising a differentiation medium for differentiation and expansion of a multicellular aggregation in suspension derived from human pluripotent stem cells that has been induced to differentiate to an artificial tissue structure, said medium comprising a basal medium for animal or human cells, wherein said differentiation medium has a viscosity between 1.7 mPa*s and 1500 mPa*s.
